(19) United States
(12) Patent Application Publication
(10) Pub. No.: US 2013/0123858 A1
(43) Pub. Date: May 16, 2013

(54) MULTIAXIAL PEDICLE ATTACHMENT DEVICE FOR VERTEBRAL OSTEOSYNTHESIS

(71) Applicant: David Attia, Montelimar (FR)

(72) Inventor: David Attia, Montelimar (FR)

(73) Assignee: SMARTSPINE, Saint-Victoret (FR)

(21) Appl. No.: 13/643,053

(22) PCT Filed: Feb. 16, 2011
(86) PCT No.: PCT/FR2011/000096

§ 371 Date: Jan. 7, 2013

(30) Foreign Application Priority Data
Apr. 23, 2010 (FR) 1001743

(51) Int. Cl. A61B 17/86

(52) U.S. Cl. CPC A61B 17/8605
USPC 606/305

(57) ABSTRACT

A multiaxial pedicle screw for vertebral osteosynthesis, including: a pedicle screw comprising a spherical head having a recess for the rotation of said pedicle screw; a socket that is hinged onto the spherical head of the pedicle screw and is provided with two diametrically opposed side grooves for receiving a connection rod; a tightening means for maintaining the position of the connection rod that is engaged into said socket; and a locking means that is positioned between said spherical head of the pedicle screw and the socket. The locking means is formed of a ring, at least the base or distal portion of which consists of a cold-compactible material having a resilient deformation capacity. Said locking means enables the locking of the socket in a desirable direction relative to the spherical head of the pedicle screw according to the position of the pedicle screw after the anchoring thereof.

### FIELD OF THE INVENTION

The present invention relates to the field of vertebral surgery, and more specifically, a pedicle attachment device for vertebral osteosynthesis.

Vertebral osteosynthesis consists of immobilising or more generally interconnecting at least two adjoining vertebrae, to correct vertebral column deformations. This operation requires, depending on the surgical procedure to be carried out, the attachment of various types of bone-anchored implants such as pedicle screws associated with connection rods to be attached to the vertebrae, by means of a connector (clamps, hooks, attachment rings, caps, connection head, etc.).

The present invention relates more specifically to a pedicle attachment device using a multiaxial pedicle screw.

### STATE OF THE PRIOR ART

Recently, “multiaxial” pedicle screws, i.e. enabling, prior to tightening, hinging of a connection head, also referred to as a “socket”, with respect to a spherical head of the pedicle screw envisaged to engage with the bone have been widely available on the market. Indeed, the multiaxial feature is suitable for easier positioning of a connection rod engaging with pedicle screws via sockets, particularly if these screws are not perfectly aligned and display differences in angulation. The surgical procedure is thus easier and the operating time reduced.

However, one problem of this multiaxial feature is that for corrections of deformations such as vertebral subsidence, deviation or rotations, generally performed with non-multiaxial screws, the mobility of the “screw-socket” system prior to tightening, impedes any adjustment of the screw for correcting the position of one vertebra with respect to the other or several others, and the realignment of the entire vertebral column.

To remedy these deficiencies or drawbacks, numerous systems for locking the multiaxial feature of the pedicle screw have been proposed. Such devices are, for example, particularly described in the documents U.S. Pat. No. 5,554,157, U.S. Pat. No. 5,549,608, U.S. Pat. No. 5,586,984, U.S. Pat. No. 6,280,442, U.S. Pat. No. 6,053,917, U.S. Pat. No. 5,964,760, U.S. Pat. No. 6,010,503, U.S. Pat. No. 5,910,142, U.S. Pat. No. 6,063,089.

The prior art particularly includes the document US 2009/0018591 describing a multiaxial pedicle screw device comprising a threaded cross-section and a spherical head with a recess for the actuation thereof, a removable socket temporarily locking on the pedicle screw, and a connection rod attached independently to the socket, wherein said socket consists of an outer socket body and an inner socket element. An attachment ring coupled with the socket is intended to hold same on the spherical head thus blocking any rotating movement. Moreover, this device comprises a cap provided with a plurality of inner protuberances for connecting by nesting with the outer socket body so as to compress the socket and attach the connection rod engaged therein

However, although this device suggests locking the multiaxial feature, it is difficult to use on the patient during the procedure. Indeed, this type of device is very complex and costly due to the high number of constituent parts, and requires successive operations which are not intuitive.

Furthermore, after anchoring the screws in the pedicle of each vertebra to be attached, the connection rod which is housed in the socket tends to be expelled by the surrounding soft tissue (muscles, ligaments, etc.) or remains in position with difficulty in the case of significant deformations.

The fitting of the clamping cap is not always easy, due to a narrow field of vision with the risk of inserting soft tissue in the socket recess. The clamping cap can then be inserted skewed into the socket, giving rise to ineffective tightening of the assembly.

For this purpose, the prior art includes discontinuous threading tightening attachments suitable for clamping in a quarter-turn, but due to the design thereof, either this tightening is not sufficiently stable over time, or, on the other hand, it is not possible to loosen when the vertebrae have fused together.

Document US 2007/0225707 describes a multiaxial pedicle attachment device comprising a pedicle screw provided with a head housed in a socket provided with guiding grooves for an intervertebral rod. The head of the pedicle screw is locked in position by a locking means inserted into the socket. The intervertebral rod is positioned in the grooves, between the locking means and a cap screwed onto the upper end of the socket. To obtain satisfactory locking, the head of the pedicle screw is shaped with concentric sharp edges situated opposite a concave lower portion of the locking means made of a plastically deformable material. In this way, when tightening the device, the edges of the screw head engage in the soft deformable material of the lower portion of the locking means. However, such a device proves to be difficult to disassemble, or reposition during a procedure. Furthermore, the inner surface of the concave lower portion of the locking means is hemispherical, such that it has a larger diameter than the head of the pedicle screw, giving rise to a larger size.

Moreover, the prior art includes a pedicle attachment device described in the document WO 2009/015100, wherein the device comprises a pedicle screw having a spherical head housed in a cavity of a socket, where the head is held in position by a locking collar provided with a plurality of flexible arms encompassing the spherical head and inserted between the head and the cavity wall. The collar is axially movable in the socket cavity, between a position with no contact with the cavity wall, and a locking position wherein the ends of the flexible arms are compressed between the cavity wall and the spherical head of the pedicle screw. By the very operating principle thereof, this device involves a significant radial size. Moreover, the socket is provided with grooves for guiding an intervertebral rod bearing on an upper portion of the collar. A clamping cap is used to secure the device in position, by pressing on the rod via a block. The device thus consists of five parts, not including the rod. Furthermore, it is not suitable for holding the rod in position with respect to the collar, before the subassembly consisting of cap and the block are fitted and tightened. The document WO 2008/124772 describes a pedicle attachment device which is relatively similar to the above device and involves the same drawbacks.

### DESCRIPTION OF THE INVENTION

The aim of the invention, considered in the various aspects thereof, is particularly that of remedying all or some of the drawbacks of the prior art. More specifically, one aim of the invention is that of providing a pedicle attachment device for easily locking the socket in a desired direction on the spherical head of the multiaxial pedicle screw after the anchoring thereof, which is suitable for adjusting the vertebrae requiring instrumentation directly.

A further aim of the invention is that of providing such a pedicle attachment device suitable for holding the connection rod in the socket.

In particular, one aim of the invention is that of providing such as a pedicle attachment device suitable for long-term locking of the assembly over time and easy unlocking when required.

A further aim is that of providing such a pedicle attachment device which is simple to use on a patient.

According to a first aspect of the invention, the invention relates to a multiaxial pedicle attachment device for vertebral osteosynthesis comprising:
a pedicle screw that is to be implanted into the bone portion of a vertebra and comprises a spherical head having a recess for the rotation of said pedicle screw;
a socket that is hinged onto the spherical head of the pedicle screw and is provided with two diametrically opposed side grooves for receiving a connection rod;
tightening means for maintaining the position of the connection rod that is engaged into said socket; and
a locking means positioned between said spherical head of the pedicle screw and the socket comprising at least one base consisting of a cold-compressible material having a resilient deformability for insertion between the spherical head of the pedicle screw and the socket and locking the socket in a desirable direction relative to the spherical head of the pedicle screw according to the position of the pedicle screw after the implantation of the pedicle screw.

The resilient deformability of the base enables the reversibility of the locking of the socket, by applying pressure on the top thereof, making the screw multiaxial again.

The socket is preferentially made of one piece, such that the device consists of only four parts, i.e, the pedicle screw, the socket, the locking means and the tightening means.

Preferably, the locking means is made entirely of a cold-compressible material.

According to one embodiment, the locking means is entirely made of a material with resilient deformability.

According to one embodiment, the base or the distal portion thereof in contact with the spherical head of the screw per se, inserted between the spherical head and the inner surface of the back of the socket, consists of a lip which becomes progressively thinner in the direction of the distal end of said locking means.

Advantageously, the base of the locking means may have a spherical inner surface.

According to a further embodiment, the locking means is made of a material having a hardness coefficient less than 82 HRB to enable the deformation thereof under the effect of compression applied by the tightening means, wherein the deformed locking means is suitable for locking the socket on the spherical head of the pedicle screw in one direction and thus locks the multiaxial feature thereof, such that correction adjustments on the vertebrae are easier.

The chosen material thus has resilient deformability due to the selected hardness thereof. In this way, the multiaxial feature of the “screw-socket” assembly is locked and the surgeon can carry out the procedures to instrument the vertebrae easily and safely.

Preferentially, the locking means comprises, in the proximal portion thereof, two diametrically opposed grooves, said grooves defining two extensions, have a curved profile rising at the sides, so as to retain the connection rod in the socket merely by clamping and locking, thus preventing said connection rod from being expelled by the surrounding soft tissue. In this way, the connection rod is positioned directly in the socket and held in position by the extensions due to the deformation of the material, avoiding the need for further procedures and materials by the surgeon.

According to one embodiment, the locking means is made of a titanium with a hardness coefficient less than 82 HRB.

According to one embodiment, the titanium of which said locking means is made has a hardness coefficient between 50 HRB and 82 HRB.

According to one particular embodiment, the tightening means consists of a quarter-turn attachment comprising a cylindrical body comprising an outer face provided with two asymmetrical fins wherein the distal end is arranged to separate the side walls of the socket, and to fit therein, the cylindrical body comprising an upper face provided with a recess for rotating the quarter-turn attachment, by means of a suitable tool.

According to one particular embodiment of the invention, the tightening means is shaped to simultaneously lock in position the socket on the spherical head of the pedicle screw and the connection rod in the socket. The surgeon can lock the entire attachment device in a single procedure, thus reducing the operating time. The attachment remains in place over time, without requiring forced tightening and once the vertebrae have fused, the attachment can be released and removed without catching.

According to a further aspect of the invention, the invention relates to a multiaxial pedicle attachment device for vertebral osteosynthesis comprising:
a pedicle screw that is to be implanted into the bone portion of a vertebra and comprises a spherical head having a recess for the rotation of said pedicle screw;
a socket comprising:
   side walls defining two diametrically opposed side grooves for receiving a connection rod, wherein the side walls have concave side faces for mounting, and
   a back acting as a lower cavity for housing a lower portion of the head of the pedicle screw and a bearing surface for the lower portion of the head of the pedicle screw;
a locking means or means, comprising a base having a concave wall acting as an upper cavity arranged opposite the lower cavity of the socket to come into contact with an upper portion of the head of the pedicle screw, wherein the concave wall of the base is shaped for insertion between the upper portion of the spherical head of the pedicle screw and the concave faces for fitting the side wall of the socket and for resilient deformation by locking the socket in a desirable direction with respect to the spherical head of the pedicle screw after implanting the pedicle screw.

By means of the resiliently deformable base of the locking means, locking in position of the spherical head of the pedicle screw is obtained very simply by clamping the base between the concave mounting faces of the side wall of the socket and the head of the pedicle screw. The surgical procedure to obtain this locking is very simple since it is simply necessary to insert the locking means to bear against the head of the pedicle screw while holding the socket in position. Conversely, unlocking is obtained very easily by pressing on the socket, for example by means of a light axial impact.

This locking device is particularly suitable for a pedicle screw wherein the head is spherical, making it possible to produce multiaxial pivoting of the socket very simply with respect to the head of the pedicle screw under optimal reliability conditions.

In this case, it is advantageous to envisage that the bearing surface of the lower cavity of the back of the socket is within a hemispherical geometric envelope having a radius of curvature equal to the radius of the spherical head of the pedicle screw. This lower cavity acts as a basin forming a swivel joint with the spherical head.

For the same reasons, it is advantageous to envisage that the concave wall of the locking means is within a hemispherical geometric envelope having a radius of curvature equal to the radius of the spherical head of the pedicle screw.

According to one embodiment, the concave wall of the base of the locking means is positioned so as to come into contact with an upper portion of the head of the pedicle screw along a contact surface including at least one circular contact line situated on a contact cone centred on the centre of the spherical head of the pedicle screw and having a working angle of less than 150°. This ensures that the contact loads between the locking means and the screw have a component along the axis of the contact cone. Preferentially, the contact cone has a working angle less than 120°, increasing the axial component of the resultant of the contact forces between the locking means and the head of the pedicle screw.

According to one embodiment, the locking means in contact with the head of the pedicle screw is entirely situated in a cone centred on the centre of the spherical head of the pedicle screw and having a working angle less than 175° and preferably less than 170°. In other words, the locking means encompasses the head of the pedicle screw on at least one hemisphere, ensuring easy assembly and disassembly. Moreover, this arrangement limits the radial size of the device.

Preferentially, the mounting faces of the side walls of the socket are within the same cylindrical or truncated geometric envelope. The cylindrical shape is particularly advantageous since it prevents the resultant of the contact forces between the locking means and the socket having a component in a direction tending to unlock the locking means.

According to a further aspect of the invention, the invention relates to a multiaxial pedicle attachment device for vertebral osteosynthesis comprising:
a pedicle screw that is to be implanted into the bone portion of a vertebra and comprises a spherical head having a recess for the rotation of said pedicle screw;
a socket having a reference geometric axis and comprising:
   side walls defining two side grooves situated in the same reference geometric plane containing the reference geometric axis, wherein the two side grooves are diametrically opposed, and
   a back acting as a lower cavity for housing a lower portion of the head of the pedicle screw and a bearing surface for the lower portion of the head of the pedicle screw;
a locking means or part, comprising:
   a base having a concave wall acting as an upper cavity arranged opposite the lower cavity of the socket to come into contact with an upper portion of the head of the pedicle screw, and
   a proximal portion having at least two extensions defining two diametrically opposed side grooves together and aligned with the side grooves of the socket in the reference plane, wherein the extensions having contact faces opposite each other; and
a cylindrical rod inserted in the grooves of the socket and the grooves of the proximal portion of the locking means, bearing against the contact faces of the extensions along a contact interface having at least one normal perpendicular to the reference axis of the socket, and held by clamping following resilient deformation of the extensions of the proximal portion of the locking means.

The locking of the socket with respect to the head of the pedicle screw and the securing of the rod are thus obtained by means of a single part in the previous definitive tightening phase of the device. Multiple functions are thus obtained, while economising resources considerably.

According to one embodiment, the contact faces of the extensions have a curved profile, having a preferably complementary concavity with respect to that of the rod. The contact face profile may particularly be cylindrical.

According to a further aspect of the invention, the invention relates to a multiaxial pedicle attachment device for vertebral osteosynthesis comprising:
a pedicle screw that is to be implanted into the bone portion of a vertebra and comprises a spherical head having a recess for the rotation of said pedicle screw;
a socket having a reference geometric axis and comprising:
   side walls defining two side grooves situated in the same reference geometric plane containing the reference geometric axis, wherein the two side grooves are diametrically opposed and the side walls are provided with a tightening interface; and
   a back acting as a lower cavity for housing a lower portion of the head of the pedicle screw and a bearing surface for the lower portion of the head of the pedicle screw;
a locking means or part, comprising at least one base having a concave wall acting as an upper cavity arranged opposite the lower cavity of the socket to come into contact with an upper portion of the head of the pedicle screw;
a cylindrical rod inserted in the grooves of the socket; and
a tightening means or part suitable for moving from a disassembled position to a tightening position by being inserted into the tightening interface, by means of a helical movement inducing:
   separation of the side walls of the socket; and
   axial bearing on the rod inducing axial bearing of the rod on the locking means, in turn inducing resilient deformation of the base of the locking means locking the socket in position with respect to the head of the pedicle screw.

The tightening means is thus shaped to simultaneous lock in position the socket on the spherical head of the pedicle screw and the connection rod in the socket. The surgeon can lock the entire attachment device in a single procedure, thus reducing the operating time. The attachment remains in place over time, without requiring forced tightening and once the vertebrae have fused, the attachment can be released and removed without catching.

Preferentially, the tightening means is a quarter-turn attachment, suitable for tightening merely by screwing with a small angle of rotation and thus ergonomic use.

Preferentially, the tightening interface consists of thread portions.

According to one embodiment, the socket further comprises a proximal portion having at least two extensions defining two diametrically opposed side grooves together and aligned with the side grooves of the socket in the reference plane, wherein the extensions have contact faces opposite each other, and the rod inserted in the grooves of the socket is also inserted in the grooves of the proximal portion of the locking means bearing against the contact faces of the extensions along a contact interface having at least one normal perpendicular to the reference axis of the socket.

According to one embodiment, the locking means is a ring having a central recess, suitable for minimising the quantity of material required for this part, wherein the functional portions of the part are grouped together at the periphery thereof.

Preferentially, the locking means comprises a load transmission surface for coming into contact with the rod and transmitting the loads applied to the rod to the locking means so as to lock the locking means. This load transmission surface preferentially consists of the backs of the grooves of the locking means. The dimensions of the grooves of the locking means and the grooves of the socket are thus determined such that, when the rod is oriented in the groove plane, perpendicular to the reference axis of the socket and the rod is moved in the groove plane parallel to the reference axis, the rod reaches the backs of the slots of the locking means without touching the back of the grooves of the socket.

According to a further aspect of the invention, the invention relates to a pedicle attachment device comprising at least two multiaxial pedicle devices as described above, and a connection rod suitable for being inserted and locked in the sockets of the two multiaxial pedicle devices.

Preferentially, the connection rod and the locking means are shaped in a complementary manner for the rigid connection thereof by clamping and locking.

According to a further aspect of the invention, the invention relates to a pedicle attachment device for vertebral osteosynthesis comprising a pedicle screw comprising a threaded cross-section to be implanted into the bone portion of a vertebra and a spherical head provided with a recess for the rotation thereof and whereon a socket is hinged, wherein said socket is provided with two diametrically opposed side grooves for receiving a connection rod held in position by tightening means and locking means inserted between the spherical head of the pedicle screw and the socket. The locking means consist of a ring wherein at least the base is made of a cold-compressible material, wherein the locking means are suitable for locking the socket in a desirable direction with respect to the spherical head of the screw, according to the position of the pedicle screw after the anchoring thereof.

Preferably, this ring is made entirely of a cold-compressible material.

According to one embodiment, the ring or at least the base thereof is made of a material with resilient deformability.

### BRIEF DESCRIPTION OF THE FIGURES

The aims, features and advantages above, along with others, will emerge more clearly from the following description and the appended figures, wherein:

FIG. 1 is an exploded external view of the pedicle attachment device according to the invention, illustrating the pedicle screw rigidly connected to a socket, a connection rod and the tightening means.

FIG. 2 is an exploded external view of the pedicle attachment device representing the pedicle screw, the socket and the locking means schematically, according to the present invention

FIG. 3 is an axial sectional view illustrating the pedicle attachment device prior to the positioning of the tightening means.

FIG. 4 is a similar axial sectional view to the previous figure, showing the insertion of the ring between the spherical head and the socket using the tightening means.

FIG. 5 is a perspective view of the pedicle attachment device showing a single screw thereof, according to the present invention.

FIG. 6 is a top view of the tightening means, according to the present invention.

FIG. 7 is an axial sectional view of the attachment device fitted on two adjacent vertebrae.

### DESCRIPTION OF ONE EMBODIMENT OF THE INVENTION

Reference is made to said figures to describe interesting, but non-limiting, examples of embodiments of the multiaxial pedicle screw and the pedicle attachment device, according to the invention.

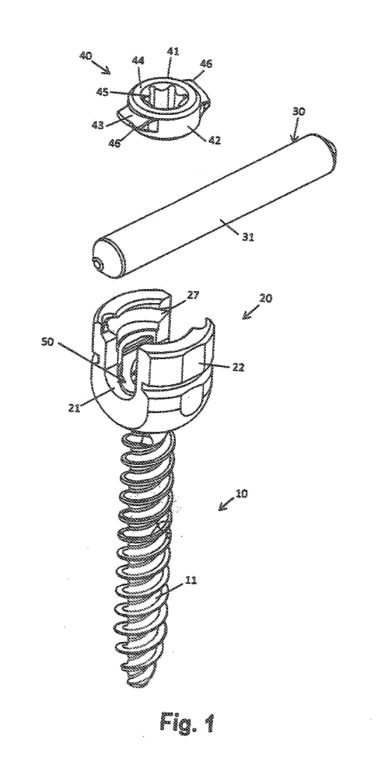

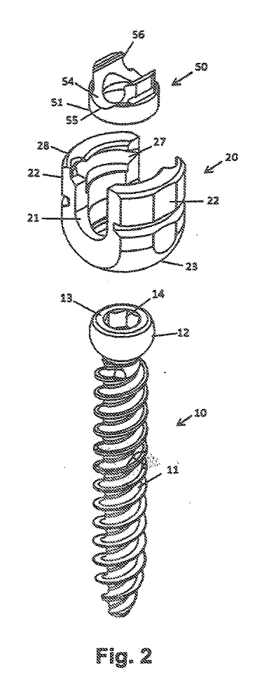

Throughout the text, the term “socket” adopted by orthopaedic surgery professionals and orthopaedic material suppliers refers to a connection head in the form of a socket suitable for receiving a connection rod and rigidly connected to a pedicle screw. With reference to FIGS. 1 and 2, it can be seen that the pedicle attachment device for vertebral osteosynthesis consists of a bone-anchored pedicle screw 10 on the spherical head 12 whereof a socket 20 is hinged, a connection rod 30 and tightening means 40 for assembling the connection rod 20 to the screw 10 via said socket 20. The pedicle screw 10 has at least one threaded portion 11 for anchoring same in the bone portion of a vertebra. Preferably, it is threaded all along the length of the rod thereof. It comprises a spherical head 12 comprising a recess 14 on the upper face 13 thereof, to rotate the pedicle screw 10 in the bone portion with a suitable screwing tool. Preferably, the threaded cross-section 11 comprises double threading. The socket 20 consists of a hollow body 28 having a general cylindrical shape, two U-shaped diametrically opposed side grooves 21, extending parallel to the axis of said body 28, said grooves defining two vertical faces 22 for receiving a cylindrical connection rod 30 and a back 23 comprising at the centre thereof an opening 24 wherein the spherical head 12 is permanently housed. Depending on the embodiment illustrated, the spherical head 12 is held in the cavity 25 provided in the back 23 of the socket 20 after the pedicle screw 10 has been inserted into the opening 24. The socket 20 is thus swivel-mounted on the spherical head 12, in a multiaxial manner due to the dimensions of the cavity 25 and those of the spherical head 12.

The upper inner walls 26 of the vertical faces 22 of the socket 20 defined by the side grooves 21 comprise a thread 27 for rotating and screwing by means of the tightening means 40 for locking the connection rod 30 inserted in the socket 20 in position.

According to the example of an embodiment shown, the tightening means 40 consist of a quarter-turn attachment comprising a cylindrical body 41 comprising on the outer face 42 thereof two asymmetrical and diametrically opposed fins 43 in the form of a thread segment wherein the thread height is extended, giving rise to retentive tightening in a quarter-turn and also easier loosening. The cylindrical body 41 has on the upper face 44 thereof and at the centre of said body, a recess 45 for rotating same in the socket 20 using a suitable tool (not shown), such as a screwdriver or tightening wrench.

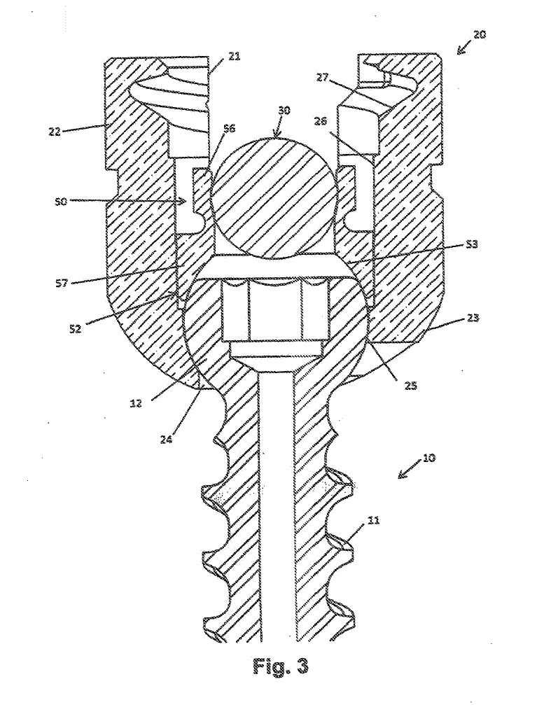

The distal ends 46 of the fins 43, which are substantially bevelled, separate the inner walls 26 of the socket 20 during the rotation of the quarter-turn attachment 40 and are fitted in said walls 26 so as to lock said fins 43 of the quarter-turn attachment 40 preventing spontaneous and untimely loosening thereof. The multiaxial pedicle screw 10, according to the invention, comprises, as can be seen in FIGS. 2 and 3, and according to one characteristic arrangement, a locking element consisting of a locking ring 50 comprising a hollow cylindrical body 51, complementary with the inner wall 26 of the socket 20 and, sized to fit therein before and after deformation. This ring 50 may be provided already fitted in the socket 20 or be fitted before anchoring the pedicle screw 10 in the bone portion of a vertebra. The ring 50 may be released by applying axial pressure on the top of the socket, restoring the multiaxial feature of the screw.

The locking ring 50 comprises a base or distal portion 52 having a spherical inner surface. This base 52 consists of an annular lip 57 having an approximately triangular cross-section tapering in the direction of the lower opening 53 of said ring 50. This annular lip 57 is inserted or to be inserted between the spherical head 12 of the pedicle screw 10 and the socket 20. The base or distal portion of the ring 50 has a spherical inner surface 58. This locking ring 50 comprises in the proximal portion thereof two opposed side grooves 55 parallel to the axis thereof, and defining two side extensions 56 having a curved profile for holding the connection rod 30. The side grooves 55 of the locking ring 50 are aligned with the side grooves of the socket 20. The connection rod 30 is thus held in the ring 50 merely by clamping and locking, given the resiliency of the material of which said ring is made, this makes it possible to prevent said rod from being expelled by the surrounding soft tissue.

At least the base or distal portion 52 of the ring 50 is made of a cold-compressible material. Preferably, the ring 50 is made entirely of a cold-compressible material. According to one important characteristic arrangement, at least the base 52 of the ring 50 is made of a material with resilient deformability. Advantageously, the base 50 is made entirely of a material with resilient deformability. Preferably, the ring 50 is made of a material having a hardness coefficient of 82 according to the Rockwell ball penetrator scale (HRB), which is equivalent to a mechanical strength of 530 MPa. The material may be resiliently deformed by a force F applied by the quarter-turn attachment 40 of 320 daN. The Young's modulus of this material is 110,000 MPa and the stretchability thereof at least 20%. Due to the high degree of resilient deformability of the material, merely tightening the quarter-turn attachment 40 is suitable for creating a force F required to deform the material resiliently. The locking ring 50 fitted in the socket 20 is thus resiliently deformable under the tightening force of the quarter-turn attachment 40 in the socket 20.

The titanium of which the ring 50 is made is referred to as “soft” titanium comprising a hardness coefficient less than 82 HRB. Preferentially, this titanium may have a hardness coefficient between 50 HRB and 82 HRB. The titanium does not exhibit a rupture onset phenomenon after deformation and includes all the properties to ensure that the locking ring 50 is not harmful for the human body and also that the biological environment does not degrade said ring 50. It should be noted that another material 10 having the same specificities, mechanical features, resilient deformation and biocompatibility could be suitable for producing the ring 50.

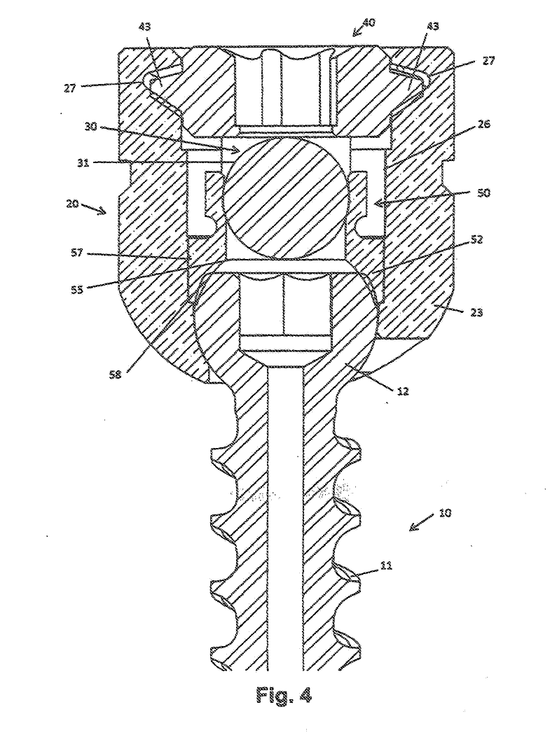

With reference to FIG. 3, the pedicle attachment device with the multiaxial pedicle screw 10 whereon the socket 20 is hinged with the ring 50 fitted therein can be seen. After fitting the pedicle screw 10 in the pedicle of a vertebra, it is possible to insert the connection rod 30 via the side grooves 21; the rod being held in the functional position by means of the side extensions 56 deformed by said rod 30. Once the socket 20 has been oriented in the desired angular position, the quarter-turn attachment 40 is screwed into the socket 20. The quarter-turn attachment 40 engages, via the two asymmetrical fins 43 thereof in the form of a thread segment, with the thread 27 provided in the inner walls 26 of the socket 20. As can be seen in FIG. 4, during tightening, the quarter-turn attachment 40 comes into contact with the surface 31 of the connection rod 30 which, bearing on the back 54 of the side grooves 55 of the ring 50, engages the spherical portion base 52 on the spherical head 12 until the ring 50 is deformed resiliently and inserted by fitting between the spherical head 12 and the back 23 of the socket 20.

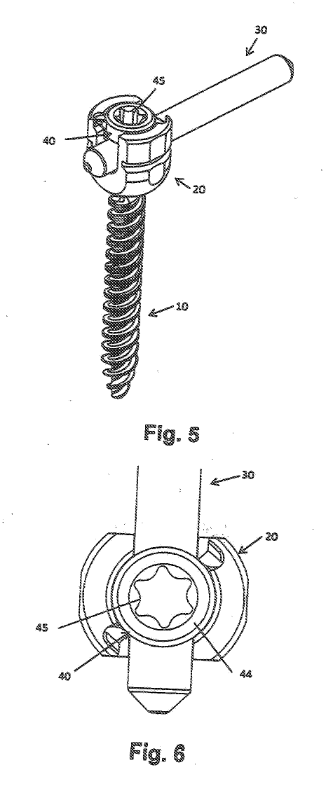

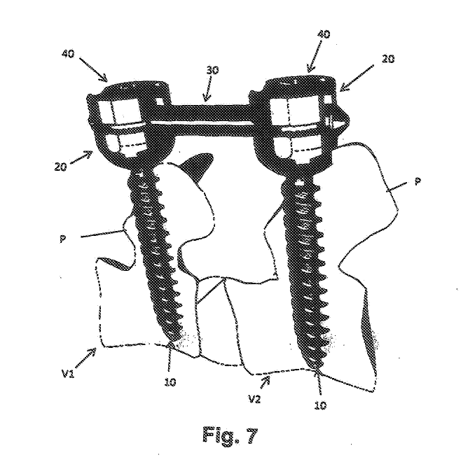

When the quarter-turn attachment 40 reaches the final position thereof, the connection rod 30 is locked in rotation and translation in the socket 20 and the spherical head 12 is locked in position in the socket 20, thus locking any rotating movement between same, removing the multiaxial feature of the pedicle screw 10. FIGS. 5 to 7 show the pedicle attachment device in the locked position thereof. In FIG. 7, two pedicle screws 10 are anchored in the pedicle P or bone portion of two adjoining vertebrae V1 and V2 with a non-rigid assembly. Since the multiaxial feature of the screws is locked, the vertebrae can be instrumented without any interfering movements on the socket 20 on the spherical head 12 of the pedicle screws 10.

The invention also relates to the pedicle attachment device using at least two multiaxial pedicle screws 10 as described above, and a connection rod 30 suitable for being inserted and locked in the sockets 20 of these multiaxial pedicle screws 10. According to one preferred embodiment, the pedicle attachment device comprises at least two multiaxial pedicle screws 10 and a connection rod 30, wherein the connection rod 30 and the two locking rings 50 are shaped in a complementary manner for rigidly connecting same by clamping and locking. It is obvious that the invention is not limited to the embodiments described as examples, but it may be used in any application covered by the claims.

1. A multiaxial pedicle attachment device for a vertebral osteosynthesis, the pedicle device comprising:
a pedicle screw to be implanted into a bone portion of a vertebra and comprising a spherical head having a recess for the rotation of said pedicle screw;
a socket hinged onto the spherical head of the pedicle screw and provided with two diametrically opposed side grooves for receiving a connection rod;
tightening means for maintaining in a determined position the connection rod engaged into said socket; and
locking means positioned between said spherical head of the pedicle screw and the socket, the locking means comprising at least one base made of a cold-compressible material having a resilient deformability for insertion between the spherical head of the pedicle screw and the socket and for locking the socket in a suitable direction relative to the spherical head of the pedicle screw after the implantation of the pedicle screw.

2. The multiaxial pedicle device according to claim 1, wherein the locking means has at least one of the following respective specificities:
the locking means is made entirely of said cold-compressible material, and, the locking means is made of a material having a resilient deforability.

3. (canceled)

4. The multiaxial pedicle device according to claim 1, wherein the locking means has a proximal portion having two diametrically opposed side grooves, wherein the side grooves of the locking means are aligned with the side grooves of the socket, the side grooves of the locking means defining two extensions having a curved profile rising laterally and retaining the connection rod in the socket by clamping and locking.

5. The multiaxial pedicle device according to claim 1, wherein the tightening means defines a quarter-turn attachment comprising a cylindrical body having an outer face provided with two asymmetrical fins having distal ends arranged to separate from each other the side walls of the socket and to fit in the side walls of the socket, the cylindrical body having an upper face provided with a recess for rotating the quarter-turn attachment.

6. The multiaxial pedicle device according to claim 1, wherein the base of the locking means has at least one of the following respective specificities:
said base defines an annular lip which becomes progressively thinner in the direction of a distal end of said base, and, sad base has an spherical inner surface.

7. (canceled)

8. The multiaxial pedicle device according to claim 1, wherein the locking means of the base is made of a material having a hardness coefficient less than 82 HRB.

9. (canceled)

10. (canceled)

11. The multiaxial pedicle device according to claim 1, wherein the tightening means is shaped to simultaneously lock in a determined position the socket on the spherical head of the pedicle screw and the connection rod in the socket.

12. The multiaxial pedicle device according to claim 1, wherein the tightening means defines a quarter-turn attachment comprising a cylindrical body having an outer face provided with two asymmetrical fins having a distal end arranged to separate from each other the side walls of the socket and to fit in the side walls of the socket, the cylindrical body having an upper face provided with a recess for rotating the quarter-turn attachment, by means of a suitable tool.

13. The multiaxial pedicle attachment device according to claim 1, wherein
the side walls define the two side grooves, wherein the side walls have concave side faces for mounting, and a back defining a lower cavity for housing a lower portion of the head of the pedicle screw and a bearing surface for a lower portion of the head of the pedicle screw;
the base having a concave wall defining an upper cavity arranged opposite the lower cavity of the socket to come into contact with an upper portion of the head of the pedicle screw, wherein the concave wall of the base is shaped for insertion between the upper portion of the head of the pedicle screw and the concave side faces for fitting the side wall of the socket.

14. The multiaxial pedicle device according to claim 13, wherein the mounting faces of the side walls of the socket are located within a common same cylindrical or truncated geometric envelope.

15. A multiaxial pedicle attachment device for a vertebral osteosynthesis, the pedicle device comprising:
a pedicle screw to be implanted into a bone portion of a vertebra and comprising a spherical head having a recess for the rotation of said pedicle screw;
a socket having a reference geometric axis and comprising:
side walls defining two side grooves situated in a common reference geometric plane containing the reference geometric axis, wherein the two side grooves are diametrically opposed,
a back defining a lower cavity for housing a lower portion of the head of the pedicle screw and a bearing surface for the lower portion of the head of the pedicle screw;
a locking means comprising:
a base having a concave wall defining an upper cavity arranged opposite the lower cavity of the socket to come into contact with an upper portion of the head of the pedicle screw, and
a proximal portion having at least two extensions defining two diametrically opposed side grooves together and aligned with the side grooves of the socket in the reference geometric plane, wherein the extensions have contact faces opposite each other; and
a cylindrical rod inserted in the side grooves of the socket and the side grooves of the proximal portion of the locking means, bearing against the contact faces of the extensions along a contact interface having at least one normal axis perpendicular to the reference geometric axis of the socket and held by clamping following a resilient deformation of the extensions of the proximal portion of the locking means.

16. The multiaxial pedicle device according to claim 15, wherein the contact faces of the extension have a curved profile.

17. A multiaxial pedicle attachment device for a vertebral osteosynthesis, the pedicle device comprising:
a pedicle screw to be implanted into a bone portion of a vertebra and comprising a spherical head having a recess for a rotation of the pedicle screw;
a socket having a reference geometric axis and comprising:
side walls defining two side grooves situated in a common reference geometric plane containing the reference geometric axis, wherein the two side grooves are diametrically opposed and the side walls are provided with a tightening interface;
a back defining a lower cavity for housing a lower portion of the head of the pedicle screw and a bearing surface for the lower portion of the head of the pedicle screw;
a locking means having at least one base having a concave wall defining an upper cavity arranged opposite the lower cavity of the socket to come into contact with an upper portion of the head of the pedicle screw;
a cylindrical rod inserted in the side grooves of the socket; and
a tightening means adapted to move from a disassembled position to a tightening position by being inserted into the tightening interface, through a helical movement inducing:
a separation of the side walls of the socket;
an axial bearing on the rod inducing an axial bearing of the rod on the locking means, which induces a resilient deformation of the base of the locking means locking the socket in a determined position with respect to the head of the pedicle screw.

18. The multiaxial pedicle device according to claim 17, wherein the tightening means has at least one of the following respective specificities:
the tightening means is a quarter-turn attachment and,
the tightening interface is defined by thread portions .

19. (canceled)

20. The multiaxial pedicle device according to claim 17, wherein the socket further comprises a proximal portion having at least two extensions defining two diametrically opposed side grooves together and aligned with the side grooves of the socket in the reference geometric plane, wherein the extensions have contact faces opposite each other, and the rod inserted in the side grooves of the socket is also inserted in the side grooves of the proximal portion of the locking means bearing against the contact faces of the extensions along a contact interface having at least one normal axis perpendicular to the reference geometric axis of the socket.

21. The multiaxial pedicle device according to claim 17, wherein the head of the pedicle screw has a spherical shape and the bearing surface of the lower cavity of the back of the socket is located within a hemispherical geometric envelope having a radius of curvature equal to a radius of the spherical head of the pedicle screw.

22. (canceled)

23. The multiaxial pedicle device according to claim 21, wherein the concave wall of the locking means has at least one of the following specificities:
the concave wall is located within a hemispherical geometric envelope having a radius of curvature equal to a radius of the spherical head of the pedicle screw, and,
the concave wall is positioned to come into contact with an upper portion of the head of the pedicle screw along a contact surface including at least one circular contact line situated on a contact cone centered on a center of the spherical head of the pedicle screw and having a working angle of less than 150°.

24. (canceled)

25. (canceled)

26. The multiaxial pedicle device according to claim 21, wherein the locking means which is in contact with the head of the pedicle screw has at least one of the following specificities:
the locking means is entirely situated in a cone centred centered on a center of the spherical head of the pedicle screw and having a working angle less than 175° and preferably less than 170° and,
the locking part has a load transmission surface for coming into contact with the rod and transmitting, to the locking means, loads applied to the rod, so as to lock the locking part.

27. (canceled)

28. (canceled)

29. A pedicle attachment device comprising at least two multiaxial pedicle devices for a vertebral osteosynthesis, each pedicle device comprising:
a pedicle screw to be implanted into a bone portion of a vertebra and comprising a spherical head having a recess for the rotation of said pedicle screw;
a socket hinged onto the spherical head of the pedicle screw and provided with two diametrically opposed side grooves for receiving a connection rod;
tightening means for maintaining in a determined position the connection rod engaged into said socket; and
locking means positioned between said spherical head of the pedicle screw and the socket, the locking means comprising at least one base made of a cold-compressible material having a resilient deformability for insertion between the spherical head of the pedicle screw and the socket and for locking the socket in a suitable direction relative to the spherical head of the pedicle screw after the implantation of the pedicle screw, and
a connection rod inserted and locked in the sockets of the at least two multiaxial pedicle devices.

30. A pedicle attachment device according to claim 29, wherein the connection rod and the locking means are shaped in a complementary manner for a rigid connection one with the other, by clamping and locking, and,
wherein the locking means has a proximal portion having two diametrically opposed side grooves which are aligned with the side grooves of the socket, the side grooves of the locking means defining two extensions having a curved profile rising laterally and retaining the connection rod in the socket by clamping and locking.
